(19) United States
(12) Patent Application Publication
(10) Pub. No.: US 2026/0110888 A1
(43) Pub. Date: Apr. 23, 2026

(54) VISUALISATION ASSEMBLY FOR MICROSURGERY

(71) Applicant: Carl Zeiss Meditec AG, Jena (DE)

(72) Inventors: Michael TOTZECK, Schwaebisch Gmuend (DE); Christoph HAUGER, Aalen (DE)

(21) Appl. No.: 19/148,467

(22) PCT Filed: Dec. 29, 2023
(86) PCT No.: PCT/EP2023/088028

§ 371 Date: Jul. 16, 2025

(30) Foreign Application Priority Data
Jan. 18, 2023 (DE) 10 2023 101 104.9

(51) Int. Cl. G02B 21/00; A61B 90/00; G02B 21/22; G02B 21/36; G02B 27/28

(52) U.S. Cl. CPC G02B 21/0012; A61B 90/37; G02B 21/0092; G02B 21/22; G02B 21/367; G02B 27/283; A61B 2090/371

(57) ABSTRACT

A visualisation assembly for microsurgery has an imaging device, an illumination device, and a polarisation determination assembly. The polarisation determination assembly has at least two video cameras designed to capture light waves of a plurality of light wavelengths in the visible wavelength range, and at least one further video camera. Each of the three video cameras is assigned an individual partial beam path in the beam path. The polarisation determination assembly has a plurality of polarisation filter devices; and an evaluation device. At least one polarisation filter device is arranged in each beam path in front of three of the at least three video cameras. The polarisation filter devices differ from one another in their polarisation effect. The evaluation device is designed to generate images with polarisation contrast by the images captured by the video cameras and to display said images by the imaging device.

The present invention relates to a visualization arrangement for microsurgery, for example for a surgical microscope, having an imaging device for creating an image representation with polarization contrast, for example a stereoscopic imaging device for creating a stereoscopic image representation with polarization contrast. The invention also relates to a method for creating an image representation with polarization contrast, for example a stereoscopic image representation with polarization contrast, of an object to be imaged, by means of a visualization arrangement for microsurgery. The invention moreover relates to a microscope, for example a surgical microscope.

Surgical microscopes are used in different disciplines of microsurgery, predominantly in neurosurgery, spine surgery, ENT surgery and eye surgery. They are distinguished in particular by stereoscopic imaging with large working distances (200 mm-600 mm) and moderate magnifications (up to approximately 20×). These properties allow the use of surgical instruments in the sterile operating field with a stereoscopic view of the operating site at the same time. In recent years, there has been a shift from analog to digital systems. The latter make use of cameras and 3-D monitors or other 3-D reproduction systems (head-mounted displays (HMDs), digital eyepieces (binocular ocular monitors—boom)) for image recording and image representation.

An essential driver for the development of surgical microscopes is the desire of users for tissue differentiation. Depending on discipline and application, this may be the distinction between tumor and healthy tissue, the distinction between white and gray matter in the brain, the improved representation of vessels and nerves, the visualization of phase objects in cataract operations (lens, capsular bag, etc.), or the visualization of membranes when performing procedures on the retina. Digital surgical microscopes offer great advantages for improved tissue differentiation because the utilized camera systems can be used to this end.

From the user's point of view, the requirements set forth below arise, especially in the field of neurosurgery, for technology relating to tissue differentiation. (i) The various tissue types must be differentiated in real time for stereoscopic image data, i.e. without a noticeable time delay for the user, e.g. the surgeon. For digital systems, this time is in the order of <50 ms. (ii) The tissue must be differentiated over the entire operating site, which typically has a diameter of approx. 10 mm-50 mm. The resolution of the tissue differentiation should correspond to the resolution of the stereoscopic image data (analog or digital), but it may also be lower. (iii) The tissue differentiation should be intuitively interpretable by the surgeon, and the applicative meaning thereof should be backed up by clinical studies. (iv) The tissue differentiation should be able to be switched on and off as necessary. (v) The technology used for differentiating tissue must not substantially increase the costs and especially the installation size of the surgical microscope. (vi) Tissue should ideally be differentiated without the use of markers and dyes since these require a lengthy approval process and are frequently linked to side effects in patients.

The scientific literature has disclosed a large number of technologies that can be used for tissue differentiation. In this context, optical technologies have many advantages over non-optical methods such as ultrasound on account of good integrability and the contactless measurement. The most important optical technologies for differentiating tissue are the detection of fluorescence/autofluorescence (continuously and/or in time-resolved fashion), laser Doppler and laser speckle imaging, optical coherence tomography (OCT), (coherent or non-coherent) Raman spectroscopy, narrowband imaging and the detection of the effect of biological tissue on the polarization state of light. The large number of scientific publications is in contrast with only a small number of technologies realized in products; this is due to the aforementioned requirements that are difficult to satisfy.

Thus, camera-based fluorescence technologies in particular have become established both in surgical microscopes and endoscopes in recent years on account of being the best at satisfying the aforementioned requirements. Currently, these are based on the three medically approved dyes of ICG, NAF and 5-ALA. Applicatively, they are used for differentiating between tumor and non-tumor and for visualizing the blood flow. The aforementioned fluorescence technologies with the three approved dyes meet requirements (i)-(v) but not requirement (vi), i.e. they are only usable in combination with a medicament. A further disadvantage of the aforementioned fluorescence options lies in the fact that it is not possible to distinguish between all applicatively relevant tissue types. There are types of tumors that do not absorb the aforementioned dyes. Moreover, it is very important in neurosurgery to spare functions of the brain during the procedure. To this end, it is necessary to identify the fiber tracts of the brain (white matter) and distinguish these from tumors or the gray matter of the cortex. This is currently not possible with any of the aforementioned dyes. For this reason, there is a great need—especially in neurosurgery—for technology that differentiates the tissue of tumors, vessels and white and gray matter.

From among the aforementioned technologies, polarization is advantageous because it manages without dyes or medicaments and reacts sensitively to different tissue types on account of the underlying physical process. At a cellular level, tumors are unordered structures while fiber tracts in particular represent strongly ordered zones. From the literature, it is known that such structures have different outcomes in terms of their effect on the polarization of the radiated-in light.

The following section describes in detail the formalism of Stokes vectors and of the Mueller matrix for the interaction of optical elements and tissue in the operating site with partially polarized light. The Mueller matrix is the 4×4 transformation matrix for the Stokes vector of the illumination light, which, following multiplication, provides the Stokes vector at the location of the detector. Knowledge of the Mueller matrix and its 16 elements therefore contains all the information of how the object acts on partially polarized light. In the field of metrology, there are numerous polarimeters that measure the complete Mueller matrix at points or over an area (e.g. https://mountainphotonics.de/product/axo-axostep/). A Mueller polarimeter consists of the following components: light source, PSG (polarization state generator), PSA (polarization state analyzer) and (point or area) detector. In this case, PSG and PSA may be realized in different ways, for example by rotating retardation elements or in stationary fashion by way of ferroelectric elements. Regarding the general background in the art, reference is made to the following publications: W. Singer, M. Totzeck, H. Gross, Handbook of Optical Systems, Vol. 2 “Physical Image Formation”, chapter 26.2.9, pp. 475ff in the series “Handbook of Optical Systems”, H. Gross (Editor), Wiley VCH [1], H. Engstrom, “Coherency matrix polarization measurements: application to magnetooptic garnet films”, Appl. Opt. 30 (1991) 1730-1734 [4], A. Pigula, N. T. Clancy, S. Arya, G. B. Hanna, D. S. Elson in: Video-rate dual polarization multispectral endoscopic imaging, International Society for Optics and Photonics, pp. 93330N-93330N-93334 (2015) [5] and Wei Sheng et al, “Quantitative Analysis of 4×4 Mueller Matrix Transformation Parameters for Biomedical Imaging”, Photonics 2019, 6, 34; doi:10.3390/photonics6010034 [6].

The following will explain how a polarization contrast, in particular for a tissue to be examined, can be ascertained from a Stokes vector. The Stokes vector is a conventional method from polarization optics for representing partial polarization states [1]. The Stokes vector consists of 4 real components, the so-called “Stokes parameters”, the values of which can be ascertained by measuring the transmission of the light through specific polarizers.

$$\vec{S} = ( \begin{matrix}{S}_{0} \\ {S}_{1} \\ {S}_{2} \\ {S}_{3}\end{matrix} ) = ( \begin{matrix}{I}_{0} \\ {P}_{0} - {P}_{90} \\ {P}_{45} - {P}_{135} \\ {P}_{R} - {P}_{L}\end{matrix} ) \qquad ( 1 )$$

In this case, I₀ represents the overall intensity, i.e. the transmission of a neutral element, P₀ represents the transmission through a linear polarizer at 0°, P₄₅ represents the transmission through a linear polarizer at 45°, P₉₀ represents the transmission through a linear polarizer at 90°, P₁₃₅ represents the transmission through a linear polarizer at 135°, P_{R} represents the transmission through a right-hand circular polarizer and P_{L} represents the transmission through a left-hand circular polarizer.

Hence, the Stokes parameter S₀ represents the intensity of the light. S₁ specifies how great the difference is between the components of the light linearly polarized in the x-direction and in the y-direction, i.e. the components of the horizontal and vertical linear polarizations, respectively. S₂ specifies how great the difference is between the components of the light linearly polarized at 45° and 135°, i.e. the components of the diagonal linear polarizations. S₄ specifies how great the difference is between the components of the light polarized in right-hand and left-hand circular fashion, i.e. the components of the circular polarizations.

An important characterization parameter of light in tissue contrast is the degree of polarization g. This is the component of the polarized light in the overall intensity and can be calculated from formula (1) according to:

$$g = \frac{\sqrt{{S}_{1}^{2} + {S}_{2}^{2} + {S}_{3}^{2}}}{{S}_{0}} \qquad ( 2 )$$

For polarized illumination, the degree of polarization generally reduces as a result of the interaction with the tissue, inter alia as a result of multiple reflections. Since the structures frequently have a linear form (e.g. nerve fiber bundles), the linear degree of polarization gₗᵢₙ in particular is relevant

$${g}_{lin} = \frac{\sqrt{{S}_{1}^{2} + {S}_{2}^{2}}}{{S}_{0}} \qquad ( 3 )$$

Analogously, the degree of circular polarization g_{circ} is

$${g}_{c i r c} = \frac{\vert {S}_{3} \vert}{{S}_{0}} \qquad ( 4 )$$

The principle of measuring the Stokes vector is explained below. Even though 6 different polarizers and a measurement of the overall intensity have an effect on the definition of the Stokes vector, 4 polarizers are already sufficient to unambiguously measure the Stokes parameters. This is due to the fact that any sum of two orthogonal polarization states already represents the overall intensity, i.e.

$${I}_{0} = {P}_{0} + {P}_{90} = {P}_{45} + {P}_{135} = {P}_{R} + {P}_{L} \qquad ( 5 )$$

Hence, the Stokes vector (1) may also be written as follows:

$$\vec{S} = ( \begin{matrix}{S}_{0} \\ {S}_{1} \\ {S}_{2} \\ {S}_{3}\end{matrix} ) = ( \begin{matrix}{P}_{0} + {P}_{90} \\ {P}_{0} - {P}_{90} \\ {P}_{45} - {P}_{0} - {P}_{90} \\ {P}_{R} - {P}_{0} - {P}_{90}\end{matrix} ) \qquad ( 6 )$$

i.e. the Stokes vector is uniquely defined by the polarization measurements of P₀, P₄₅, P₉₀ and P_{R}.

The Mueller matrix and its information content, in particular for tissue, are explained below. The polarization effect of an object such as tissue consists of transforming an incident Stokes vector into an emerging Stokes vector. In the linear regime, this transformation is described by multiplication with a 4×4 matrix, the Mueller matrix:

$${\vec{S}}_{o u t} = M {\vec{S}}_{i n} \qquad ( 7 )$$

with the Mueller matrix

$$M = ( \begin{matrix}{M}_{00} & {M}_{01} & {M}_{02} & {M}_{03} \\ {M}_{10} & {M}_{11} & {M}_{12} & {M}_{13} \\ {M}_{20} & {M}_{21} & {M}_{22} & {M}_{23} \\ {M}_{30} & {M}_{31} & {M}_{32} & {M}_{33}\end{matrix} ) \qquad ( 8 )$$

Taken by themselves, the individual Mueller matrix elements have a simple meaning. They describe the component of the excitation of one Stokes parameter by another Stokes parameter. Thus, e.g. M₁₂ represents the excitation of S₁ by S₂.

As described in the literature, e.g. in [2], the decomposition of the Mueller matrix into elementary polarization matrices is important for describing the polarization of tissue. One such decomposition is e.g. the Lu-Chipman polar decomposition, in which a Mueller matrix is represented by a product of a depolarization Mueller matrix, a retarder Mueller matrix and a diattenuator Mueller matrix:

$$M = {M}_{D i a} {M}_{R e t} {M}_{Depol} \qquad ( 9 )$$

with the depolarization Mueller matrix

$${M}_{Depol} = ( \begin{matrix}1 & 0 & 0 & 0 \\ 0 & a & 0 & 0 \\ 0 & 0 & b & 0 \\ 0 & 0 & 0 & c\end{matrix} ) \qquad ( 10 )$$

the retarder Mueller matrix

$${M}_{R} = {M}_{L R} {M}_{C R} = ( \begin{matrix}1 & \vec{0} \\ \vec{0} & {m}_{L}\end{matrix} ) ( \begin{matrix}1 & \vec{0} \\ \vec{0} & {m}_{R}\end{matrix} ) \qquad ( 11 )$$

where \vec{0} represents a 3-component 0-vector and m_{L} and m_{R} represent the 3×3 sub-matrices of linear and circular retardance. The submatrix of the linear retarder depends on 2 parameters: the absolute value of the retardance and the orientation, i.e. the position of one of the two eigenpolarizations. The circular retarder only depends on the absolute value of the circular retardance. Hence, Mᵣ is uniquely described by only 3 scalar parameters. According to [2], the retardance values arise from

$${δ}_{L} = {cos}^{- 1} ( \sqrt{{[ {M}_{R} ( 1 , 1 ) + {M}_{R} ( 2 , 2 ) ]}^{2} + {[ {M}_{R} ( 2 , 1 ) + {M}_{R} ( 1 , 2 ) ]}^{2}} ) \qquad ( 12 )$$

$$and$$

$${δ}_{C} = {tan}^{- 1} ( \frac{{M}_{R} ( 2 , 1 ) - {M}_{R} ( 1 , 2 )}{{M}_{R} ( 1 , 1 ) + {M}_{R} ( 2 , 2 )} ) \qquad ( 13 )$$

and the diattenuator Mueller matrix

$${M}_{D} = ( \begin{matrix}1 & \vec{D} \\ \vec{D} & {m}_{D}\end{matrix} ) \qquad ( 14 )$$

with the diattenuation vector

$$\vec{D} = \frac{1}{{M}_{00}} [ \begin{matrix}{M}_{01} & {M}_{02} & {M}_{03}\end{matrix} ] \qquad ( 15 )$$

and the submatrix

$${m}_{D} = \sqrt{1 - {‖ \vec{D} ‖}^{2}} I + \frac{1 - \sqrt{1 - {‖ \vec{D} ‖}^{2}}}{{‖ \vec{D} ‖}^{2}} \vec{D} {\vec{D}}^{T} \qquad ( 16 )$$

where I represents the 3×3 unit matrix.

In addition to this known decomposition, the polarimetry literature has a whole host of other decompositions of the Mueller matrix into a function of elementary matrices [2]. The decomposition supplies a physically unique and correct representation of the Mueller matrix made of depolarization, retardance and diattenuation. However, correct performance of the decomposition also requires a measurement of the entire Mueller matrix. Should this be the case, the Mueller matrix decomposition is the preferred form of analysis.

However, the polarizers are not perfect as a rule but must themselves be described by a Mueller matrix again. However, this may be taken into account in the measuring process, as shown in [4] and described in the following. Already at the stage of measuring the Stokes vectors, consideration should be given to the fact that the available components are not ideal from a polarization-optical point of view. However, the assumption may be made that they do not depolarize. Thus, their effect on partially polarized light is described by a known, albeit not necessarily simple Jones matrix. For example, a polarizer could only lead to a degree of polarization of 0.9. Instead of incorporating this property in the error budget, it is surely more sensible to take it into account during the evaluation. According to [4], it is possible to set up a general method for measuring Stokes vectors (or polarization matrices), which assumes only that the Jones matrix of the polarization-modifying components is known but does not assume that these have a specific value. The basis of this method lies in the linearity of the polarization matrix transformation in the Jones matrix

$${\underline{P}}_{o u t} = \underline{L} {\underline{P}}_{i n} {\underline{L}}^{+}$$

where Pᵢₙ and Pₒᵤₜ represent the input and output polarization matrices and L represents the Jones matrix of the measuring system. This relation is linear in Pᵢₙ, a property that is also maintained when there is a transition to intensity.

$${I}_{o u t} = Spur ( \underline{L} {\underline{P}}_{i n} {\underline{L}}^{+} )$$

Four intensity measurements with four different L allow a linear system of equations to be set up and allow Pᵢₙ to be determined,

$$\begin{matrix}{I}_{j} = Spur ( \underline{{L}_{j}} {\underline{P}}_{in} {\underline{L}}_{j}^{+} ) , j = 1 , … , 4 \\ = {\sum }_{k = 1}^{4} {A}_{jk} {P}_{k}\end{matrix}$$

where Pₖ represents the real elements P₁-P₄ of the polarization matrix. Written as matrices, the solution arises by inverting the coefficient matrix

$$\vec{I} = \underline{A} \vec{P} \vec{P} = {\underline{A}}^{- 1} \vec{I}$$

To determine the coefficients Aⱼₖ, the equation for Iⱼ must be multiplied out.

$$\begin{matrix}{I}_{j} = Spur ( \underline{{L}_{j}} {\underline{P}}_{in} {\underline{L}}_{j}^{+} ) \\ = Spur \{ ( \begin{matrix}{L}_{x x} & {L}_{x y} \\ {L}_{yx} & {L}_{y y}\end{matrix} ) ( \begin{matrix}{P}_{1} & {P}_{2} + {iP}_{3} \\ {P}_{2} - {iP}_{3} & {P}_{4}\end{matrix} ) ( \begin{matrix}{L}_{x x}^{*} & {L}_{yx}^{*} \\ {L}_{xy}^{*} & {L}_{y y}^{*}\end{matrix} ) \} \\ = ( {\vert {L}_{x x} \vert}^{2} + {\vert {L}_{y x} \vert}^{2} ) {P}_{1} + ( {L}_{x x} {L}_{x y}^{*} + {L}_{x y} {L}_{x y}^{*} + {L}_{y x} {L}_{y y}^{*} + {L}_{y y} {L}_{y x}^{*} ) {P}_{2} \\ + i ( {L}_{x x} {L}_{x y}^{*} - {L}_{x y} {L}_{x y}^{*} + {L}_{y x} {L}_{y y}^{*} - {L}_{y y} {L}_{y x}^{*} ) {P}_{3} + ( {\vert {L}_{x y} \vert}^{2} + {\vert {L}_{y y} \vert}^{2} ) {P}_{4}\end{matrix}$$

Hence, the elements of the coefficient matrix arise as

$${A}_{j 1} = {\vert {L}_{xx}^{j} \vert}^{2} + {\vert {L}_{yx}^{j} \vert}^{2}$$

$${A}_{j 2} = 2 Re \{ {L}_{xx}^{j} {L}_{xy}^{j *} + {L}_{yx}^{i} {L}_{yy}^{j *} \}$$

$${A}_{j 3} = - 2 Im \{ {L}_{xx}^{j} {L}_{xy}^{j *} + {L}_{yx}^{i} {L}_{yy}^{j *} \}$$

$${A}_{j 4} = {\vert {L}_{xy}^{j} \vert}^{2} + {\vert {L}_{yy}^{j} \vert}^{2}$$

For the real measurement, the four different measured Jones matrices must be realized by polarization-optical components. There are various options to this end. A standard method is specified in [4], wherein use is made of a linear polarizer at 0°, 45° and 90°, and a quarter wave plate with the slow axis at 90°, followed by a polarizer at 45°. An alternative measuring method uses a rotating quarter wave plate, followed by a polarizer. Naturally, even more configurations are conceivable. In the case of a “good” measuring method, the accuracy of the measurement now no longer depends on how precisely the components maintain a predetermined value (e.g. retardance, extinction ratio) but only on how well this value is known. A further important variable for the measurement accuracy is the question of how well the components span an orthonormal basis for the Jones vectors. In this case, the relative error of the Stokes vector components is introduced directly into the Mueller matrix. However, given an accuracy of the components, the relative error will become larger as the associated basis vector realized by the measuring method (i.e. the sensitivity of the measuring method for a given Stokes vector component) becomes smaller.

DE 10 2017 100 904 A1 describes an image conversion module for a microscope that is designed for a polarization measurement by means of a polarization mask. DE 102 42 983 A1, CN 107490851 A, DE 10 2018 110 806 A1 and WO 2016 170 816 A1 describe surgical microscopes that are designed for at least partial polarization determination.

The most significant challenges arising in the integration of a complete Mueller polarimeter into a surgical microscope or, in general, into a microscope requiring real-time evaluation are the requirements of (i) real-time capability, (ii) stereoscopy and (v) little installation space and low costs (see above). For this reason, surgical microscopes have to date only realized simple crossed linear polarizers in relation to illumination and observation (see below). The literature has described various approaches for polarimeters as regards the achievement of the aforementioned requirements, but without success so far.

The ZEISS EXTARO surgical microscope (https://www.zeiss.de/meditec/produkte/zahnheilkunde/operationsmikroskope/extaro-300.html) makes exclusive use of linear polarizers. A first polarizer is installed so as to be capable of pivoting into and out of the illumination beam path of the surgical microscope, and two further polarizers are integrated in the two stereo beam paths so as to likewise be capable of pivoting into and out of the illumination at right angles to the polarizer. Applicatively, the polarizers are used to suppress reflections off the surface of teeth (“NoGlare Mode”).

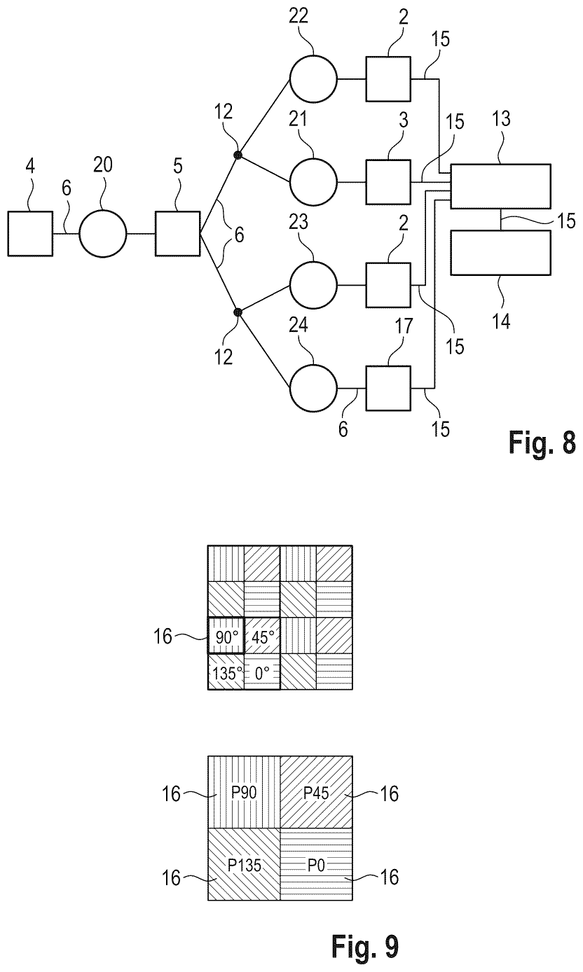

In the “3×3 Müller Polarimetric Endoscope” from FIG. 8 in J. Qi, D. S. Elson, Mueller polarimetric imaging for surgical and diagnostic application, J. Biophotonics 10, 950-982 (2017)/DOI 10.1002/jbio.201600152 [2], a linear polarizer is fixedly positioned upstream of the illumination outlet at the distal end of the endoscope. The endoscope is rotated about the optical axis and fixedly aligned in three different positions during the measuring procedure. Thus, three different alignments of the linear polarization may be realized in the illumination. In the observation beam path, a filter wheel in which 3 different polarizers are integrated is attached in front of the camera. A 3×3 submatrix of the Mueller matrix can be determined using this endoscope. The measurement duration is 11.6 s. A rotation of the endoscope during clinical use is hardly practicable.

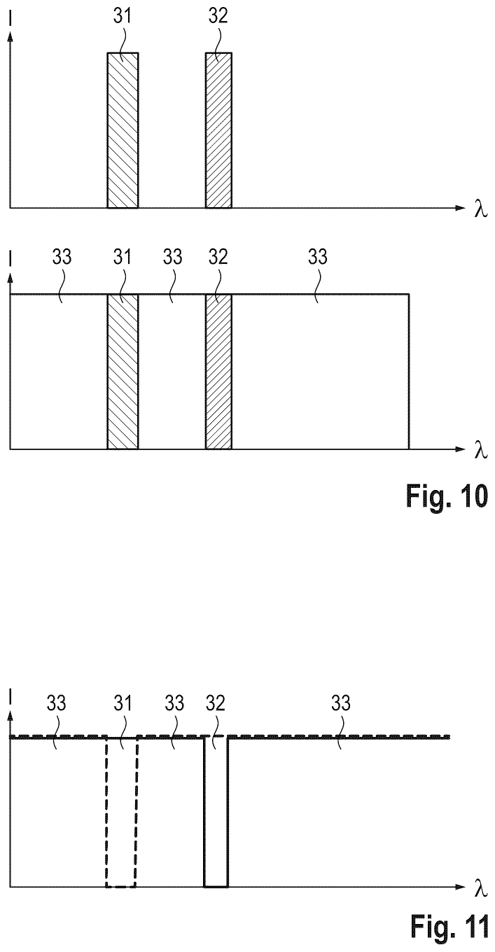

Further, [2] describes an endoscope for measuring the complete Mueller matrix with a rotating PSG and time-sequential PSA with a measurement duration of 30 s (see FIG. 10). On account of the long measurement duration and the rotating elements, this endoscope is not suitable either as a product for clinical use or any use requiring real-time evaluation. A stereo endoscope with a linear polarizer that covers the illumination and one stereo channel at the distal end is also described in [2]. The second stereo channel measures the polarization perpendicular thereto (see FIG. 11). Additionally, the polarization measurements are combined with spectral narrowband imaging. This endoscope satisfies the requirements of real-time capability and little installation space/low costs, but no stereoscopic image is created for the user, e.g. a surgeon. Moreover, only a very small part of the Mueller matrix is determined (2×2 submatrix), and so essential information about the properties of the sample is not acquired.

A “Müller Polarimetric Colposcope” with PSG and PSA based on ferroelectric modulators is described in Vizet, J., Rehbinder, J., Deby, S. et al., In vivo imaging of uterine cervix with a Mueller polarimetric colposcope, Sci Rep 7, 2471 (2017), https://doi.org/10.1038/s41598-017-02645-9 [3]. The module is fastened below the colposcope and can be pivoted in and out. The measurement duration is approximately 1.6 s; the entire 4×4 Mueller matrix is measured. In terms of optical structure, a colposcope corresponds to a surgical microscope. In this case, the polarimeter module is attached below the main objective and has a monoscopic beam path. For this reason, this approach does not attain real-time capability, stereoscopy and compact structure and low costs.

Stereoscopic tissue differentiation in real time (<50 ms) and over large observation fields (>1 cm diameter) that goes beyond the simple use of crossed polarizers is not known to date.

Against the described background, the problem addressed by the present invention is that of providing an advantageous visualization arrangement for microsurgery and an advantageous method for creating an image representation with polarization contrast by means of a visualization arrangement for microsurgery, and also a microscope, in particular a surgical microscope.

The aforementioned problems are solved by a visualization arrangement for microsurgery as claimed in claims 1 and 13, by a method as claimed in claim 15 for creating an image representation with polarization contrast by means of a visualization arrangement for microsurgery and by a microscope as claimed in claim 21. The dependent claims contain further advantageous configurations of the invention.

The visualization arrangement for microsurgery according to the invention, for example for a surgical microscope, comprises an imaging apparatus, by preference a stereoscopic imaging apparatus, an illumination device and a polarization-determining arrangement. The polarization-determining arrangement comprises at least two video cameras, which are designed to capture light waves at a plurality of light wavelengths in the visible wavelength range, i.e. in the wavelength range between 400 nm and 780 nm, and at least one further video camera, preferably two further video cameras, a plurality of polarization-filtering devices and an evaluation device. An individual partial beam path in the beam path is assigned to each of the three aforementioned video cameras. Thus, the three aforementioned video cameras may be arranged next to one another in the beam path or, in other words, arranged parallel to one another in relation to the beam path. In this context, this does not necessarily mean a spatially or geometrically parallel arrangement or a spatial arrangement next to one another but an arrangement that allows the separate capture of light waves in different partial beam paths of a beam path by the individual video cameras.

The at least two video cameras for capturing multicolored visible light are RGB cameras, for example. The at least one further video camera might be a fluorescence camera, for example.

At least one polarization-filtering device is arranged in the beam path upstream of each of three of the at least three video cameras, for example upstream of each of the three aforementioned video cameras. Optionally, at least one polarization-filtering device may be arranged in the beam path downstream of the illumination device and upstream of an object space region, for example an object to be imaged or an object plane. The polarization-filtering devices are set or settable such that the polarization filters arranged in the beam path upstream of the at least one specified further video camera and at least one of the aforementioned video cameras designed to capture light waves at a plurality of light wavelengths in the visible wavelength range differ from one another in terms of their polarization effect. Thus, at least two, preferably three polarization-filtering devices are set or settable in a manner deviating from one another upstream of the video cameras. For example, the polarization-filtering devices may also be set or settable such that the three polarization filters arranged in the beam path upstream of the three aforementioned video cameras differ from one another in terms of their polarization effect, i.e. such that each of the three aforementioned video cameras receives light simultaneously with the other two video cameras, with said received light differing from that of the other video cameras in terms of its polarization.

In the present case, an object to be imaged is also understood to be a subject or a partial region of a subject, for example human or animal or plant tissue.

The evaluation device is designed to create image representations, for example two- or three-dimensional image representations, with polarization contrast using the images captured by the video cameras and to display, preferably stereoscopically display, said image representations by means of the imaging apparatus. In the process, the created image representations with polarization contrast may be superimposed on the beam path of the imaging apparatus. They may be overlaid in partially transparent fashion on an image representation without polarization contrast. Thus, the created image representations with polarization contrast may be capable of being superimposed and masked. In the process, the polarization-filtering devices may be capable of being switched on and off.

In an advantageous variant, the evaluation device is designed to display the created image representations with polarization contrast by means of the imaging apparatus in a manner overlaid with white-light image representations captured by means of at least two video cameras, which are designed to capture light waves at a plurality of light wavelengths in the visible wavelength range. The at least two video cameras might be the aforementioned at least two video cameras. However, more than the three aforementioned video cameras may also be present. In particular, it might be the case that no polarization-filtering device is arranged, at least sequentially, in front of at least one of the video cameras for capturing multicolored visible light. In this way, high-resolution multicolored image representations can be created by means of at least one of the video cameras for capturing multicolored visible light, and the created image representations with polarization contrast can be overlaid on said high-resolution multicolored image representations. For example, the polarization-filtering device may be switchable in such a way in front of at least one of the video cameras for capturing multicolored visible light that image representations with an upstream polarization filter and without an upstream polarization filter are captured in alternation-preferably at intervals of less than 100 ms, in particular less than 40 ms. As a result, high-resolution image representations of the object to be imaged can be captured with and without polarization contrast using the same video camera. In an alternative, at least one additional video camera may be present, wherein at least one video camera is designed to capture, without polarization contrast, a multicolored image representation of the object to be imaged. An image representation with polarization contrast may be displayed in a manner overlaid on the captured image representation.

For example, white light may be understood to mean light with a broadband color spectrum. In this case, the wavelengths may lie substantially in the visible range. The distribution of the wavelengths is usually continuous. A white color impression arises for humans on account of the broadband color spectrum of a “white-light source”.

In this case, the color impression arising in the eye is relevant. Thus, this is a physiological effect and not a physical effect. That is to say, a white color impression may be based on different spectra that are restricted to the visible range.

The visualization arrangement may be configured as a visualization arrangement of a microsurgical apparatus, for example as a visualization arrangement of a surgical microscope, in particular of a surgical microscope designed for neurosurgical operations. The visualization arrangement preferably has a fully digital configuration.

Should the visualization arrangement be configured as a component of a microscope, the microscope may be configured as a fully digital microscope, in particular a surgical microscope. The visualization arrangement may be designed to stereoscopically record and display white light video data and/or fluorescence video data in real time, in particular to display said data in real time with a created image representation with polarization contrast overlaid thereon. In the case of an application as a component of a surgical microscope, the latter may preferably be designed for neurosurgical operations. It may have an object surface or object plane with an extent or a diameter of between 10 mm and 50 mm. By preference, the resolution of the created image representations with polarization contrast is lower than the resolution of a (stereoscopic) image representation without polarization contrast that is displayed by means of the (stereoscopic) imaging apparatus, or the resolutions correspond to one another. For example, the aforementioned resolutions may deviate from one another by less than 10 percent.

In a further variant, the respective meaning of the ascertained polarization contrast may be stored applicatively for the specific application and may be able to be switched on and off in the form of a display. For example, the type of tissue imaged with polarization resolution or features of the tissue derivable from the polarization-resolved image representation may be displayed in the case of a neurosurgical application. To this end, the complete Mueller matrix for individual types of tissue may be determined in clinical studies. The clinically relevant elements, or in general the elements relevant to the respective application, may be derived for individual tissue types from an analysis of the Mueller matrix. Within the scope of using the visualization arrangement, e.g. within the scope of a microscope, it is then possible to only determine the relevant elements of the Mueller matrix or measure the Stokes vector components required to this end. The visualization arrangement may be designed to determine a fixed reduced Mueller matrix.

An advantage of the visualization arrangement according to the invention is that it offers an integrated polarization measuring technique that meets the requirements in respect of real-time capability, small installation space, low costs and optionally stereoscopy. In a variant as visualization arrangement of a surgical microscope, the present invention allows improved tissue differentiation, especially in the field of neurosurgery. By means of the visualization arrangement according to the invention, it is possible to simultaneously capture data that allow the determination of the Stokes vectors and the complete Mueller matrix or of Mueller matrix elements or Mueller matrix coefficients that are essential to the respective application.

In an advantageous variant, at least one of the polarization-filtering devices, for example the at least three, e.g. four, polarization-filtering devices arranged upstream of the three, e.g. four, video cameras, by preference all of the aforementioned polarization-filtering devices, comprise(s) a plurality of polarizers that differ from one another, for example at least four polarizers that differ from one another. At least one of the polarization-filtering devices may comprise a filter wheel for switching between a plurality of different polarizers. It is thus possible to introduce a respective polarizer from the plurality of polarizers into the beam path. For example, at least one of the polarization-filtering devices may comprise at least three, in particular four, linear polarizers and optionally at least one circular polarizer. To this end, the at least one polarization-filtering device of said polarization-filtering devices may comprise one or more retardation elements and/or at least variable retarders and/or ferroelectric elements, etc.

In an advantageous setting, i.e. in the case of a suitable selection of the polarizers of the individual polarization-filtering devices introduced into the beam path, of a variant with four cameras, at least 3 polarizers, preferably all 4 polarizers, of the four polarizers arranged upstream of the video cameras differ from one another with respect to their polarization effect. For example, 3 of the polarizers can be linear polarizers, for example a selection of the P0, P45, P90 and P135 polarizers shown in FIG. 3, and 1 polarizer can be a circular polarizer, or 3 of the polarizers or 4 of the polarizers can be linear polarizers. Filters that are identical in terms of the polarization effect may be arranged or set upstream of the video cameras designed to capture light waves at a plurality of light wavelengths in the visible wavelength range. By preference, their polarization effect is orthogonal to the polarizer arranged optionally downstream of the illumination device and upstream of an object to be imaged.

In an advantageous variant, at least one, for example two, of the at least one further video cameras are designed to capture monochrome light, i.e. light at a defined wavelength or in a defined wavelength range, in particular fluorescence. In this case, the capturable wavelength range may also be located beyond the visible wavelength range.

In a further variant, at least one of the aforementioned video cameras, e.g. the three aforementioned video cameras, may take the form of a polarization camera. For example, the polarization camera may comprise 4 linear polarizers at the pixel level. The polarization camera may comprise at least one circular polarizer, for example instead of one of the 4 linear polarizers, i.e. 3 linear polarizers and one circular polarizer, at the pixel level. A polarization-filtering device that is arranged upstream of a video camera that does not take the form of a polarization camera, i.e. upstream of a video camera designed to capture light waves at a plurality of light wavelengths in the visible wavelength range, may comprise a circular polarizer or be set as a circular polarizer. This may be realized in the form of a combination of a linear polarizer and a retarder, e.g. a quarter wave plate element at zero degrees.

The evaluation device is advantageously designed to determine a set, in particular a finite set, of Mueller matrix coefficients by means of the images captured by the video cameras and determine, for example calculate, a polarization contrast derived therefrom.

In a particularly advantageous configuration, the polarization-filtering device optionally arranged in the beam path downstream of the illumination device and upstream of the object space region is designed to simultaneously polarize at least two defined, mutually deviating wavelengths, for example two defined, mutually deviating wavelength ranges, in a manner deviating from one another, i.e. differently. This enables a simultaneous acquisition of a relatively large amount of data, by means of which it is possible in turn to determine or ascertain a relatively large number of Mueller matrix coefficients. As a result, it is possible to determine stronger polarization contrasts and hence generate high-quality image representations with polarization contrast. Moreover, the simultaneously available detection channels (e.g. the number of camera sensors in RGB cameras), optionally spectrally encoded detection channels, may be used for different pieces of polarization information regarding an object to be examined. The polarizations and spectra may thus be designed such that a plurality of detection channels, e.g. each detection channel, supplies different information, e.g. tissue information.

At least one of the polarization-filtering devices may comprise a color filter or wavelength filter and/or a wavelength-selective or wavelength-specific polarizer. In particular, the above-described configuration may be realized therewith. The respective polarization-filtering device may for example comprise a notch filter, in particular a polarizing notch filter, and/or a bandpass filter, in particular a polarizing bandpass filter.

In general terms, the aforementioned polarization-filtering devices may comprise filters for fluorescence and/or polarization, in particular wavelength-specific polarization. For example, a first polarization state for at least one first wavelength or a first wavelength range and a second polarization state for at least one second wavelength or a second wavelength range may be set or settable.

The illumination device may be designed for emitting bichromatic light or polychromatic light. By preference, the illumination device is designed to emit polychromatic light, and the polarization-filtering device arranged in the beam path downstream of the illumination device and upstream of the object space region comprises at least one, for example two, notch filters, preferably polarizing notch filters, and/or at least one, for example two, bandpass filters, preferably polarizing bandpass filters.

Furthermore, the visualization arrangement may comprise at least one beam splitter, for example a dichroic beam splitter. The latter may be arranged in the beam path between the object space region and the video cameras.

At least one of the video cameras designed to capture or receive light waves at a plurality of light wavelengths in the visible wavelength range may be configured as a 3-chip camera or as a 1-chip camera.

A visualization arrangement according to the invention for microsurgery that is an alternative to the above-described visualization arrangement comprises an imaging apparatus, e.g. a stereoscopic imaging apparatus, an illumination device and a polarization-determining arrangement. The polarization-determining arrangement comprises two polarization cameras, with each of the two polarization cameras being assigned an individual partial beam path in the beam path, and an evaluation device. The evaluation device is designed to create image representations with polarization contrast using the images captured by the polarization cameras and to display said image representations, preferably stereoscopically, by means of the imaging apparatus. The alternative visualization arrangement according to the invention offers a solution that is equivalent to that of the above-described visualization arrangement according to the invention. Both variants use the same technical effects for creating image representations with polarization contrast and have the same features and advantages-which were described in detail above. In particular, the alternative visualization arrangement according to the invention may include the aforementioned features and properties that are optional in the context of the visualization arrangement according to the invention described first.

By preference, the evaluation device is designed to display the created image representations with polarization contrast in a manner overlaid with white-light image representations, with the white-light image representations being captured by means of the two polarization cameras. In this case, the display is implemented by means of the imaging apparatus.

The method according to the invention for creating an image representation with polarization contrast, e.g. a stereoscopic image representation with polarization contrast, of an object by means of an above-described visualization arrangement according to the invention for microsurgery, in particular for a surgical microscope, comprises the following steps: Light waves, in particular in the visible wavelength range and optionally therebeyond, are radiated onto the object by means of the illumination device. Light waves, which are emitted by the object following an interaction of the object with the radiated-in light waves, are captured or received by means of the at least two polarization cameras or at least three video cameras. An image representation with polarization contrast, preferably a stereoscopic image representation with polarization contrast, is created on the basis of a generated polarization state of the radiated-in light waves and/or an analyzed polarization state of the captured or received light waves. The method according to the invention has the already described features and advantages of the visualization arrangement according to the invention.

In an advantageous variant, light waves in a defined polarization state are radiated-in by means of the illumination device and a polarization-filtering device arranged in the beam path downstream of the illumination device and upstream of the object. The polarization state of the received light waves may be analyzed by means of the aforementioned video cameras and the polarization-filtering devices arranged upstream of said video cameras in the beam path. The evaluation device can be used to create an image representation, e.g. a stereoscopic image representation, with polarization contrast on the basis of an analyzed polarization state of the captured or received light waves, wherein a set of Mueller matrix coefficients is determined by means of the images captured by the video cameras and/or the polarization cameras, and a polarization contrast derived from said coefficients is determined, in particular calculated.

The image representation, e.g. stereoscopic image representation, with polarization contrast is preferably created within a time interval of less than 50 ms. The resolution of the created image representation, e.g. stereoscopic image representation, with polarization contrast deviates from the resolution of a corresponding image representation, e.g. stereoscopic image representation, without polarization contrast by preferably less than 10 percent. Particularly preferably, the image representation, e.g. the stereoscopic image representation, with polarization contrast is created within a time interval of less than 50 ms and is displayed in a manner overlaid on a multicolored image representation, e.g. a stereoscopic image representation.

Photogrammetry may be applied to the video signals within the scope of the method according to the invention, in order to identify respective corresponding pixels, e.g. in the stereo channels, for the polarimetric evaluation. In the process, a numerical value that reflects a polarization property may be calculated pixel-by-pixel. From this, a color value that reflects the polarization property may be derived pixel-by-pixel. In particular, there can be a stereoscopic representation of the polarization property, either on its own or in a manner overlaid on the white-light image, in real time.

The microscope according to the invention which may be a surgical microscope—for example a neurosurgical surgical microscope—comprises an above-described visualization arrangement according to the invention and/or is designed to carry out an above-described method according to the invention. It has the features and advantages already specified above. The microscope according to the invention is preferably configured stereoscopically and/or partly or fully digitally.

The microscope, in particular the surgical microscope, may comprise an optics unit with a variable focal length, i.e. an optics unit with a variable focus (varioscope), and/or at least one objective, which is arranged in the beam path upstream of at least one of the video cameras, and/or at least one zoom optics unit and/or a surroundings camera, i.e. a video camera for capturing a spatial region around an object region to be imaged microscopically.

The invention is explained in detail below on the basis of exemplary embodiments with reference to the accompanying figures. Although the invention is more specifically illustrated and described in detail by means of the preferred exemplary embodiments, nevertheless the invention is not limited by the examples disclosed, and other variations can be derived therefrom by a person skilled in the art, without departing from the scope of protection of the invention.

The figures are not necessarily accurate in every detail and to scale and can be presented in enlarged or reduced form for the purpose of better clarity. For this reason, functional details disclosed here should not be understood to be limiting, but merely to be an illustrative basis that gives guidance to a person skilled in this technical field for using the present invention in various ways.

The expression “and/or” used here, when it is used in a series of two or more elements, means that any of the elements listed can be used alone, or any combination of two or more of the elements listed can be used. For example, if a structure is described containing the components A, B and/or C, the structure can contain A alone; B alone; C alone; A and B in combination; A and C in combination; B and C in combination; or A, B, and C in combination.

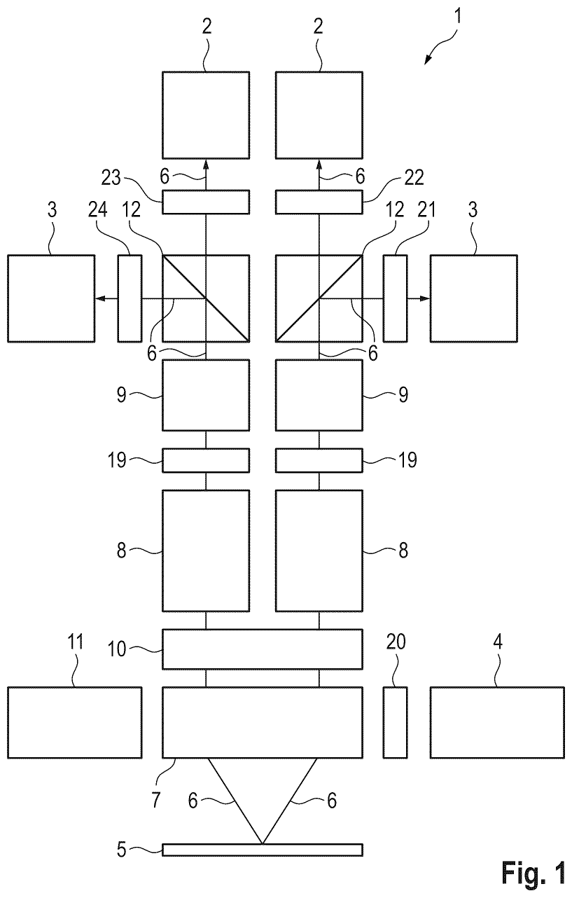

FIG. 1 schematically shows, in the form of a block diagram, a surgical microscope according to the invention having a visualization arrangement according to the invention.

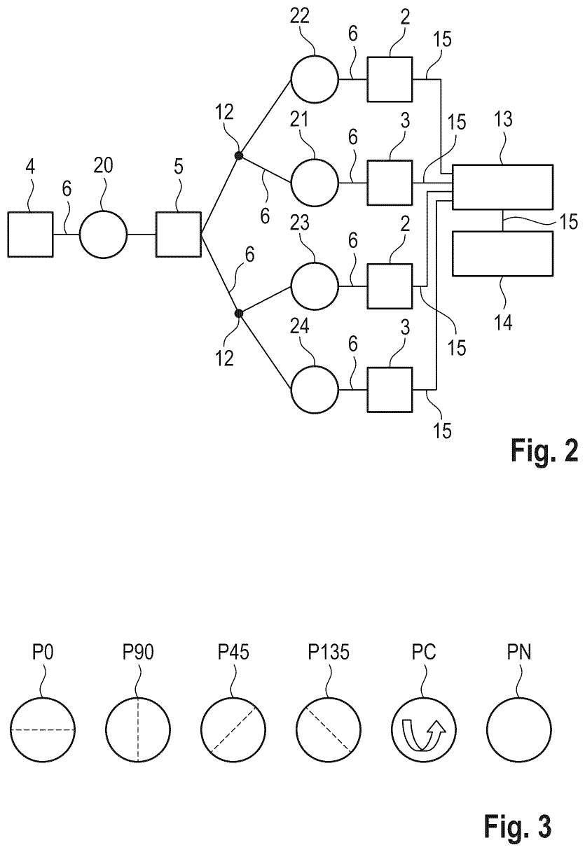

FIG. 2 schematically shows, in the form of a block diagram, the polarization-optical mode of operation of a visualization arrangement according to the invention.

FIG. 3 schematically shows exemplary polarization filters.

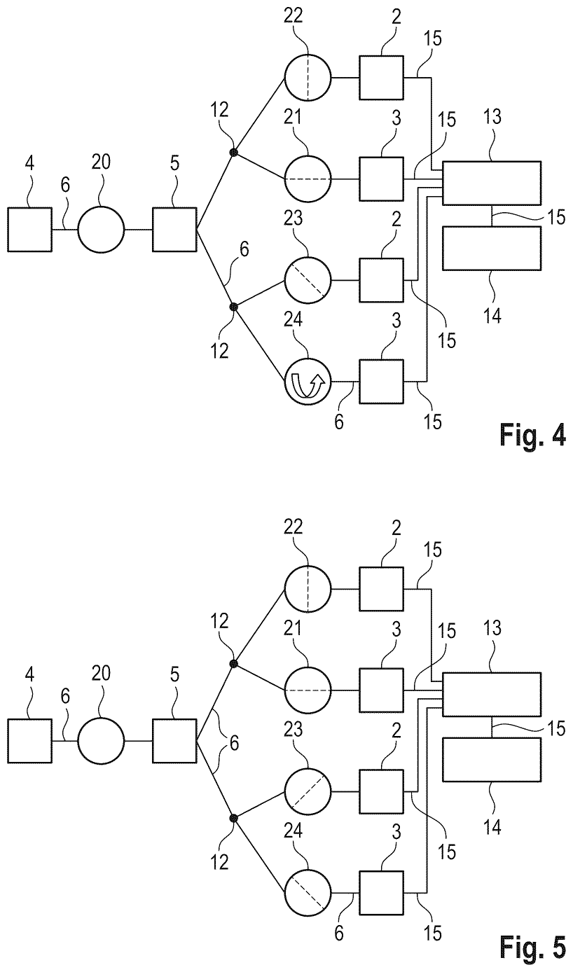

FIG. 4 schematically shows, in the form of a block diagram, the polarization-optical mode of operation of an exemplary variant of a visualization arrangement according to the invention.

FIG. 5 schematically shows, in the form of a block diagram, the polarization-optical mode of operation of an exemplary variant of a visualization arrangement according to the invention.

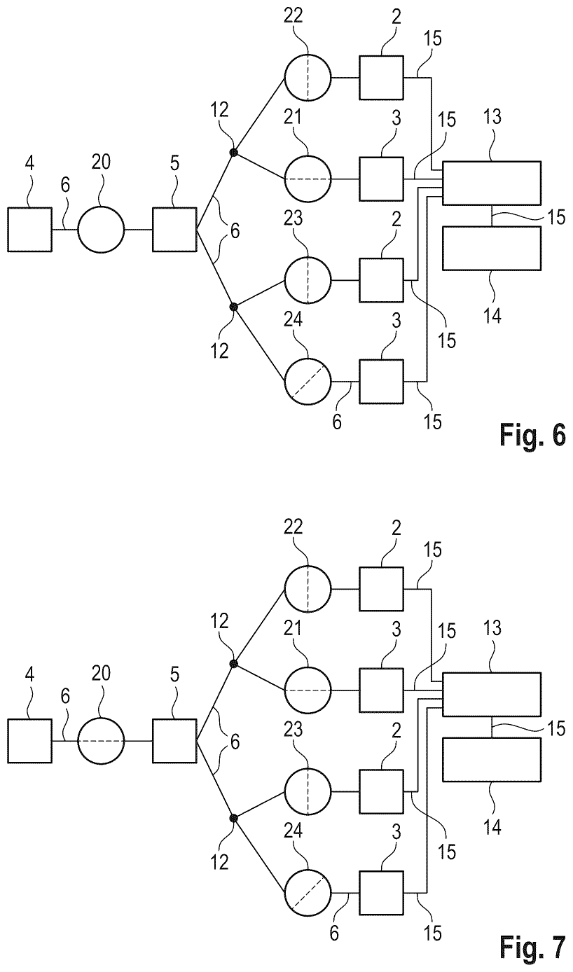

FIG. 6 schematically shows, in the form of a block diagram, the polarization-optical mode of operation of an exemplary variant of a visualization arrangement according to the invention.

FIG. 7 schematically shows, in the form of a block diagram, the polarization-optical mode of operation of an exemplary variant of a visualization arrangement according to the invention.

FIG. 8 schematically shows, in the form of a block diagram, the polarization-optical mode of operation of an exemplary variant of a visualization arrangement according to the invention.

FIG. 9 schematically shows the principle of a polarization camera.

FIG. 10 schematically shows two diagrams that represent possible illuminations of the object region.

FIG. 11 schematically shows a diagram that illustrates the realization of an illumination as shown in FIG. 10, bottom, by means of polarizing notch filters.

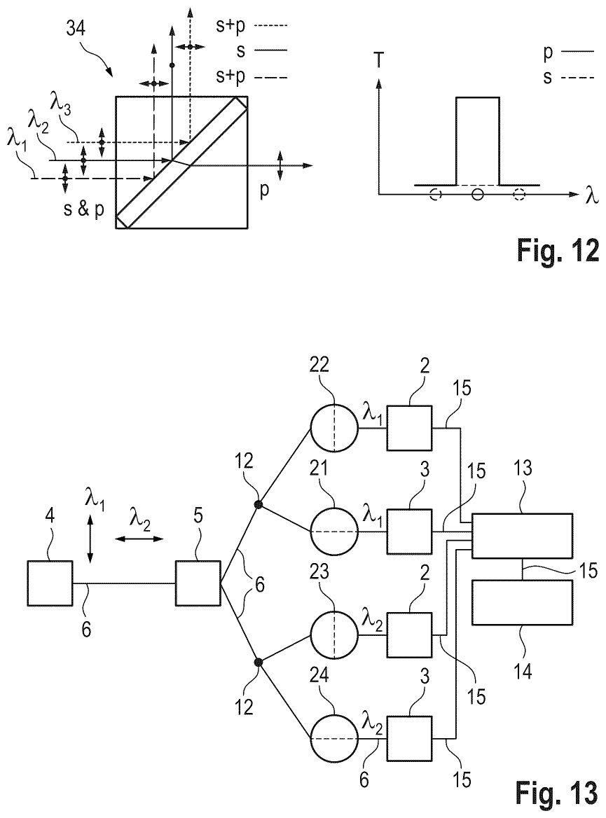

FIG. 12 schematically shows a realization of the variant shown in FIG. 11 by means of polarizing bandpass filters.

FIG. 13 schematically shows, in the form of a block diagram, the polarization-optical mode of operation of an exemplary variant of a visualization arrangement according to the invention using two different wavelengths.

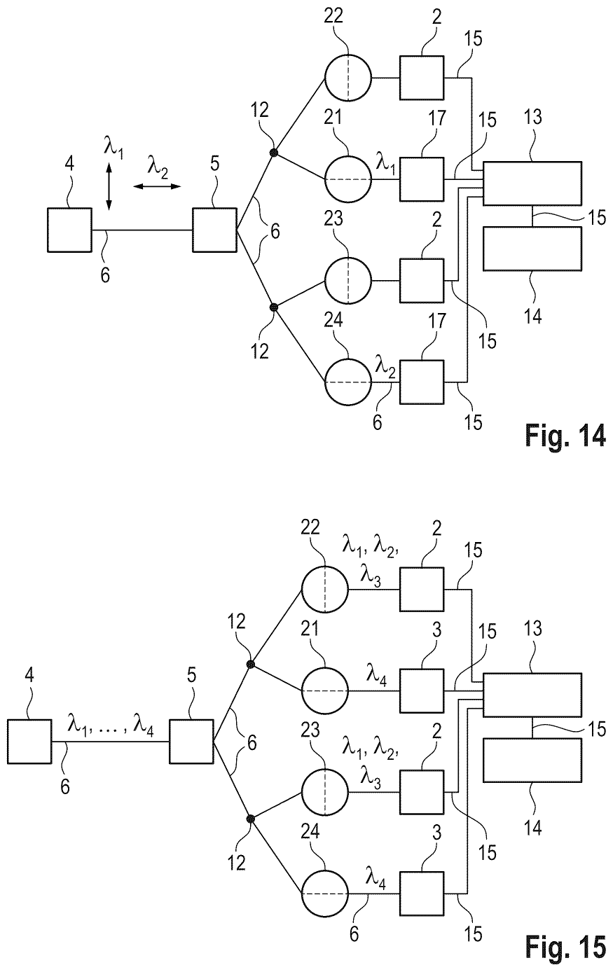

FIG. 14 schematically shows, in the form of a block diagram, the polarization-optical mode of operation of an exemplary variant of a visualization arrangement according to the invention using two different wavelengths and two polarization cameras.

FIG. 15 schematically shows, in the form of a block diagram, the polarization-optical mode of operation of an exemplary variant of a visualization arrangement according to the invention using two different wavelengths.

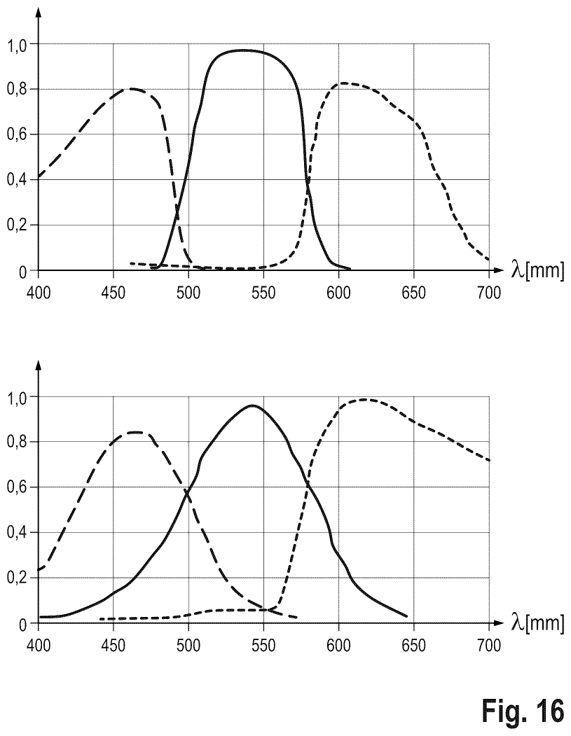

FIG. 16 schematically shows spectral resolutions of a prism and of a filter.

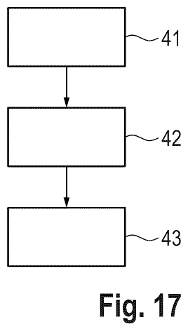

FIG. 17 schematically shows a method according to the invention in the form of a flowchart.

In the form of a block diagram, FIG. 1 schematically shows a surgical microscope 1 according to the invention. For example, the surgical microscope 1 may be designed for application within the scope of neurosurgery and spine surgery. The surgical microscope 1 comprises a visualization arrangement having an illumination device 4, an object plane or an object space region 5, in which an object to be imaged in magnified fashion may be arranged for example, a stereoscopic imaging apparatus and a polarization-determining arrangement. The illumination device 4 is designed to illuminate the object space region 5, i.e. to radiate light waves onto an object. The beam path is identified by arrows with the reference sign 6.

By preference, the microscope 1 takes the form of a fully digital microscope. It is preferably designed to record and display both white-light video data and fluorescence video data stereoscopically in real time. This is achieved by the use of in each case two video cameras 2 designed to capture light waves at a plurality of light wavelengths in the visible wavelength range (VIS cameras, e.g. RGB cameras) and at least one further video camera 3, preferably two further video cameras 3 as shown, configured as monochrome cameras in order to increase the sensitivity and designed to capture fluorescence (fluorescence cameras). The two VIS cameras 2 are either 3-chip or 1-chip cameras by preference. The fluorescence cameras 3 may also be designed to capture light waves beyond the visible range.

In addition, the microscope 1 contains an optics unit with a variable focal length (varioscope) 7, two zoom optics units 8, iris elements 19 and two video objectives 9. In this case, the optics unit with a variable focal length (varioscope) 7, two zoom optics units 8 and two video objectives 9 are arranged in the beam path 6 between the object space region 5 or an object arranged in the object space region and the aforementioned video cameras 2, 3. In FIG. 1, a laser autofocus apparatus 10 is arranged in the beam path 6 between the optics unit with a variable focal length 7 and the zoom optics units 8. A beam splitter 12 is arranged in each beam path 6 between the video objectives 9 and the aforementioned video cameras 2 and 3. Individual partial beam paths are created by the beam splitters 12, and so an individual partial beam path is assigned to each of the aforementioned video cameras 2 and 3 in the beam path 6. Moreover, the microscope 1 optionally contains a surroundings camera 11, with which it is possible to capture a large field around the object region captured by means of the microscope, e.g. the magnified operating site, and which can be used for tool tracking and navigation functions.

The polarization-determining arrangement comprises a plurality of polarization-filtering devices 20-24 and an evaluation device (not shown here) in addition to the aforementioned four video cameras 2, 3. In this case, a respective polarization-filtering device 21-24 is arranged in the beam path 6 upstream of each of the four aforementioned video cameras 2, 3, and a polarization-filtering device 20 is arranged in the beam path 6 downstream of the illumination device 4 and upstream of the object space region 5. In this case, the polarization-filtering devices 21-24 are set or settable such that the polarization filters 21-24 arranged in the beam path 6 upstream of at least one, preferably both, of the two specified further video cameras 3 and at least one, in particular both, of the aforementioned video cameras 2 designed to capture light waves at a plurality of light wavelengths in the visible wavelength range in each case differ from one another in terms of their polarization effect. In other words, the setting of the polarization of at least one of the polarization devices 22, 23, which is arranged upstream of one of the video cameras 2 designed to capture visible light (VIS cameras), differs during operation from the setting of the polarization of at least one polarization device 21, 24, which is arranged upstream of one of the further video cameras 3 (e.g. fluorescence cameras).

The polarization-determining arrangement enables an analysis of the polarization state of the light emitted by an object 5, wherein a plurality of polarization measurements may be performed simultaneously with the aid of the plurality of video cameras 2, 3. This allows a capture and evaluation of the necessary data in real time. To create a polarization-resolved, e.g. three-dimensional stereoscopic, overall image of an object, it is possible for example for differently set polarization filter wheels or different polarizers 20-24 to be positioned upstream of the four cameras 2, 3 and in the beam path 6 downstream of the illumination device 4. On the one hand, these are equipped with suitable excitation and observation filters for the aforementioned fluorescence options and on the other hand they contain polarizers suitable for polarimetry.

The video cameras 2 are configured as polarization cameras should an alternative visualization arrangement according to the invention as described at the outset be used. In this variant, it is possible to omit the further video cameras 3, the beam splitters 12 and the polarization-filtering arrangements 20 to 24.

Simplified diagrams are used below for the polarization-optical description of the system. FIG. 2 schematically shows, in the form of a block diagram, the polarization-optical mode of operation of a visualization arrangement according to the invention. In this case, an evaluation device 13 is used to implement an analysis and evaluation of the image data captured by means of the four cameras 2, 3 and a synthesis of said data to form a three-dimensional overall image with polarization contrast. The created overall image is visualized by means of a stereoscopic imaging apparatus 14 or displayed to a user. The data transfer between the video cameras 2, 3 and the evaluation device 13 and between the evaluation device 13 and the stereoscopic imaging apparatus 14 is identified by reference sign 15. Within the scope of analyzing and evaluating the acquired data, Stokes vectors or parts thereof are preferably ascertained and a plurality of Mueller matrix coefficients, preferably more than 4, are ascertained, in particular calculated. The polarization contrast is ascertained with the aid of the ascertained Mueller matrix coefficients.

FIG. 3 schematically shows exemplary polarization filters and their designation. In this case, the polarization effect of the filters is indicated by the direction of the transmitted polarization. The polarizers 20-24 each comprise a number, preferably a plurality, of different polarizers, for example one or more of the polarizers shown in FIG. 3. By preference, the polarization is individually settable at each of the polarization-filtering devices.

To measure the complete Stokes vector, four different polarizers are required at filter positions 21-24. An example thereof is shown in FIG. 4, wherein the polarizers may also be divided differently among the four filter positions 21-24. In surgical microscopy, it might primarily be the linear polarization component that is of interest, and so it may optionally be possible to omit the circular polarizer. Then, as shown in FIG. 5, the latter could be replaced by a 135° filter, for example, in order to obtain redundancy when determining the overall intensity.

In a further variant, which is shown in FIG. 6, the same polarization filters are arranged or set in front of the two video cameras for visible light 2 (VIS cameras or RGB cameras), in order not to interfere with the stereo imaging. In this case, it is advantageous for the illumination polarization 4, 20 to be set orthogonal to the polarization of the filter 22, 23 upstream of the two video cameras for visible light 2. This is shown in FIG. 7, wherein the Stokes vector components S0, S1 and S2 can be measured with unimpeded stereo imaging.

In a digital surgical microscope, Stokes parameters can also be measured by using one or more polarization cameras, which realize the four necessary measurements for the ascertainment of the Stokes vector by way of small polarization filters on the camera pixels. In that case, at least one of the four video cameras 2, 3 is a polarization camera 17. By preference, one of the further video cameras 3 is a polarization camera, for example as illustrated in FIG. 8. In this context, the polarization-filtering arrangement 24 of FIG. 8 is optional. The principle of a polarization camera is shown in FIG. 9. Four pixels 16 arranged adjacently in a plane are each provided with a polarizer that deviates from each of the other polarizers.

Available polarization cameras, e.g. from Sony, have 5 megapixels and achieve 23 frames per second. This means a time delay of 43 ms between 2 images, i.e. just below the sought-after specification of 50 ms. In Equation (1), the 4 polarizers correspond to P₀, P₄₅, P₉₀ and P₁₃₅. This can measure the first 3 Stokes parameters (S₀, S₁, S₂), but not the 4th component S₃. For imaging tissue in reflection this may be an allowable restriction because the structure features are substantially linear, i.e. should also primarily influence the linear polarization components. What could become critical is that fiber layers may be located on top of one another at an oblique orientation to one another in the case of deep tissue imaging. This would correspond to a combination of differently oriented linear retardation plates, which could lead to a rotation and hence also lead to circular birefringence. To measure all Stokes parameters, it is possible for e.g. the 135° polarizer or the 45° polarizer in FIG. 5 to be overlaid with a quarter wave retarder at 0°. Together, the two then form a circular polarizer.

In the case of an incomplete measurement of the Mueller matrix, a Chipman and Lu decomposition as described at the outset is not possible. Moreover, it is not the mandatory goal of a surgical microscope with polarization contrast to completely and precisely measure the Mueller matrix, instead it should provide the user, for example the surgeon, with a good tissue contrast. In this respect, numerous analyses have been conducted in the past.

A variant is described in [6]. In it, a series of parameters that emerge immediately from the Mueller matrix coefficients have been defined on the basis of simulations of the polarization effect of isotropic and anisotropic tissue. Thus, it is sufficient in that case to measure a finite set of Mueller matrix coefficients and calculate a contrast derived therefrom. Examples to this end from [6] are summarized in the following table:

| Contrast | Calculation | |
|---|---|---|
| Linear depolarization | $b = \frac{{M}_{11} + {M}_{22}}{{M}_{00}}$ | (17) |
| Anisotropic scattering | ${t}_{1} = \frac{\sqrt{{( {M}_{11} - {M}_{22} )}^{2} + {( {M}_{12} + {M}_{21} )}^{2}}}{{M}_{00}}$ | (18) |
| Anistropic scattering | ${t}_{2} = \frac{\sqrt{{M}_{10}^{2} + {M}_{20}^{2}}}{{M}_{00}}$ | (19) |
| Birefringence | ${t}_{3} = \frac{\sqrt{{M}_{31}^{2} + {M}_{32}^{2}}}{{M}_{00}}$ | (20) |

However, these are only examples. There are further meaningful combinations of Mueller matrix elements that can be used as the image contrast of surgical microscopy.

A linearly anisotropic medium examined in [6], which in terms of its form compares well with biological fibers, exhibits the following symmetry of the Mueller matrix:

$$M = ( \begin{matrix}{I}_{0} & A & B & 0 \\ A & C & D & 0 \\ B & D & E & 0 \\ 0 & 0 & 0 & 0\end{matrix} ) \qquad ( 21 )$$

Only a 3×3 subset differs from zero and moreover is symmetric. Hence, there are only the 6 independent components I₀, A, B, C, D, E, which need to be determined.

To measure parts of the Mueller matrix, it is necessary to radiate-in not only one polarization state but several. An obvious solution would be that of configuring the filter 20 to be rotatable or complemented by a ferroelectric liquid crystal filter. However, illumination with a plurality of polarization states is only possible sequentially in time in this case. Since the polarized imaging is already only just within the sought-after specification, a time-sequential polarized illumination would probably clearly exceed the requirement of a time delay of no more than 50 ms for the tissue contrast. If time cannot be used as differentiating parameter for the polarization of the illumination, then it is possible to use the wavelength of the illumination light within the scope of the present invention. A simultaneous illumination with two polarization states becomes possible if these are arranged at different positions in the spectrum. In that case, the associated images may also be reconstructed again using a spectral filter. That is to say, the illumination must be differently polarized in two narrow spectral bands. If a “normal color impression” should nevertheless arise, unpolarized light or light in any desired polarization state should be present around said bands (see FIG. 3). Otherwise, there is the option of a bichromatic illumination. For example, this may be realized by 2 notch filters, as shown in FIG. 10.

FIG. 10 schematically shows 2 diagrams that represent possible illuminations of the object space region. Both diagrams plot the intensity I of the radiated-in light as a function of the wavelength A. The upper diagram shows a bichromatic illumination with a first wavelength range 31 and a second wavelength range 32. The two wavelength ranges 31 and 32 are polarized in a manner deviating from one another. The lower diagram shows a polychromatic illumination, wherein the two wavelength ranges 31 and 32 are polarized in a manner deviating from one another, and the remaining spectral range 33 is unpolarized. For example, this may be realized by two notch filters, as shown in FIG. 11. In this case, two narrowband polarized spectral components 31 and 32 are realized by the transmission of unpolarized light through two narrowband polarizing notch filters. To realize the concept shown in FIG. 11, it would be possible e.g. to use polarizing bandpass filters in reflection, as shown in FIG. 12. FIG. 12 illustrates the mode of operation of a polarizing bandpass filter 34 from Semrock (https://www.semrock.com/a-new-class-of-polarization-optics-designed-specifically-for-lasers.aspx). The beam path for three different wavelengths λ₁, λ₂ and λ₃ and the respective linear polarizations s and p are shown to the left. In this case, s and p identify mutually orthogonal linear polarizations. A diagram that plots the transmission T through the filter 34 as a function of the wavelength λ and the polarization s and p is shown to the right in FIG. 12.

Depending on the polarization states radiated-in and measured, different Mueller matrix components are accessible to the measurement, e.g. M₀₀, M₀₁, M₁₀, M₁₁ using the configuration shown in FIG. 13. In the variant shown in FIG. 13, wavelengths or wavelength ranges λ₁ and λ₂ that are polarized perpendicular to one another are radiated-in, wherein the individual polarized wavelengths are analyzed by different cameras, in the variant shown the wavelength λ₁ by a first video camera for visible light (RGB camera) 2 and a first further video camera 3 and the wavelength λ₂ by a second video camera for visible light (RGB camera) 2 and a second further video camera 3.

Naturally, other configurations are conceivable, e.g. the RGB cameras 2 could be replaced by monochrome cameras 3, or vice versa; however, without polarization cameras, it is possible to measure not 3 or 4 but only 2 Stokes vector components on account of the spectral division, S0 and S1 in the variant shown in FIG. 13. However, this could also be S0 and S2, rendering the Mueller matrix components M₀₀, M₀₂, M₂₀, M₂₂ accessible.

In the variant shown in FIG. 14, half of the Mueller matrix, i.e. 8 Mueller matrix elements, can be measured using 2 polarization cameras 17 as further cameras 3 and two input polarizations. In this case, the filter 20 (not plotted here) is a polarizing notch filter which only polarizes the two wavelengths λ₁ and λ₂ in an unpolarized illumination spectrum. The rest of the illumination spectrum is unpolarized. Filter 21 is a filter for the wavelength λ₁ and optionally the polarization 1, and filter 24 is a bandpass filter for the wavelength λ₂ and optionally the polarization 2. In an alternative, the neutral beam splitter could also take the form of a dichroic beam splitter, e.g. a first for a wavelength with a first polarization and a second for a wavelength with a second polarization. This would be more efficient for the light budget but is to the detriment of flexibility. The advantage of this method is that the polarization becomes an additional option that does not interfere with standard imaging.

FIG. 15 shows a further embodiment for spectral encoding, in which the spectral properties of the RGB channels of the two color video cameras 2 are advantageously used. As described in relation to FIG. 14, the filter 1 (not plotted here) is also a polarizing notch filter in FIG. 15, albeit now designed such that it is illuminated with 4 different wavelengths with defined, predetermined polarizations. The polarizations for the different wavelengths may be different or partially identical. Either 1-chip or 3-chip cameras can be used as video cameras 2 (RGB cameras). In the case of 1-chip cameras, a Bayer filter separates the one sensor into 3 spectrally separated pixel arrays, whereas the splitter prism used in 3-chip cameras allows the spectral division among the three sensors. The wavelengths of the filter 20 are selected in such a way in FIG. 15 that the RGB sensors detect the wavelengths separately, and there is no crosstalk between the various wavelengths. This can be achieved e.g. by illumination with the wavelengths of 400 nm, 540 nm, 680 nm, as exhibited by the typical spectral sensitivities of the RGB sensors and Bayer filters in FIG. 16. FIG. 16 shows the relative spectral sensitivity as a function of wavelength for a splitter prism (top) and a Bayer filter (bottom). The 4th wavelength of the illumination filter 20 is chosen such that the two monochrome cameras 3 are able to detect signals in a manner spectrally separated from the RGB cameras 2. This can be achieved by e.g. illumination with a wavelength in the infrared wavelength range, at which the RGB cameras 2 are no longer sensitive (e.g. at 800 nm). Naturally, it is also conceivable that the wavelengths are chosen differently, and interfering wavelengths are removed upstream of the sensors by suitable notch filters. The filters 22 and 23 upstream of the RGB cameras 2 are therefore ideally designed as multi-bandpass filters for the wavelengths of 400 nm, 540 nm and 680 nm. At the same time, the filters 22 and 23 may be designed to be different in relation to their polarization properties. The filters 21 and 24 upstream of the further video cameras (monochrome cameras) 3 are designed as bandpass filters for the wavelength of 800 nm, with likewise optionally different polarization properties with regards to the filters 21 and 24.

The exemplary embodiment in FIG. 15 describes a special case and can be generalized as follows: In the first step, the number N of detection channels present in the system is determined. In the example shown in FIG. 1, these are 8 channels, wherein the two further video cameras 3 (monochrome cameras) supply two channels and the two video cameras for visible light 2 (RGB cameras) additionally supply six channels. Now, the system is designed such that these N detection channels supply an optimal tissue contrast for the respective application. This is achieved by virtue of the fact that all N channels differ in at least one property, e.g. in terms of spectrum and/or polarization.

A first possible exemplary embodiment for a system with 8 detection channels is an illumination with 8 different polarizations, which are spectrally separated, and a detection with 8 detectors, which by way of one polarization analyze in spectrally separated fashion. A second possible exemplary embodiment for a system with 8 detection channels is an illumination with 2 defined polarizations, which are spectrally separated, and a detection with 8 detectors, wherein in each case 4 detectors analyze a spectral region with 4 different analyzers.

FIG. 17 schematically shows a method according to the invention for creating an image representation, preferably a stereoscopic image representation, with polarization contrast of an object by means of an above-described visualization arrangement according to the invention, for example a visualization arrangement of a surgical microscope, in the form of a flowchart. The method comprises radiating light waves onto the object by means of the illumination device in step 41, capturing light waves, which are emitted by the object following an interaction of the object with the radiated-in light waves, by means of the at least three video cameras or the at least two polarization cameras in step 42, and creating a preferably stereoscopic image representation with polarization contrast on the basis of a generated polarization state of the radiated-in light waves and/or an analyzed polarization state of the captured light waves in step 43. Regarding specific embodiment variants of the method, reference is made to the explanations given in relation to FIGS. 1 to 16.

### LIST OF REFERENCE SIGNS

1 Surgical microscope with a visualization arrangement
2 Video camera
3 Video camera
4 Illumination device
5 Object region, object
6 Beam path
7 Optics unit with a variable focal length (varioscope)
8 Zoom optics unit
9 Video objective
10 Laser autofocus apparatus
11 Surroundings camera
12 Beam splitter
13 Evaluation device
14 (Stereoscopic) imaging apparatus
15 (Image) data transfer
16 Pixel
17 Polarization camera
19 Iris element
20 Polarization-filtering device
21 Polarization-filtering device
22 Polarization-filtering device
23 Polarization-filtering device
24 Polarization-filtering device
31 Wavelength range
32 Wavelength range
33 Wavelength range
34 Polarizing bandpass filter
41 Radiating-in light waves
42 Capturing light waves, which are emitted by the object following an interaction of the object with the radiated-in light waves
43 Creating an image representation with polarization contrast on the basis of a generated polarization state of the radiated-in light waves and/or an analyzed polarization state of the captured light waves
I Intensity
T Transmission
λ Wavelength
s Linear polarization
p Linear polarization
P0 Polarizer at 0 degrees
P90 Polarizer at 90 degrees
P45 Polarizer at 45 degrees
P135 Polarizer at 135 degrees
PC Circular polarizer
PN Neutral polarizer

1. A visualization arrangement for microsurgery, comprising an imaging apparatus, an illumination device and a polarization-determining arrangement,
wherein
the polarization-determining arrangement comprises at least two video cameras for capturing multicolored visible light and at least one further video camera, with each of the three aforementioned video cameras being assigned an individual partial beam path in the beam path, a plurality of polarization-filtering devices and an evaluation device,
wherein at least one polarization-filtering device is arranged in the beam path upstream of each of three of the at least three video cameras,
wherein the polarization-filtering devices are set or settable such that the polarization filters arranged in the beam path upstream of the at least one specified further video camera and at least one of the aforementioned video cameras for capturing multicolored visible light differ from one another in terms of their polarization effect,
wherein the evaluation device is designed to create image representations with polarization contrast using the images captured by the video cameras and to display said image representations by means of the imaging apparatus.

2. The visualization arrangement as claimed in claim 1,
wherein
the evaluation device is designed to display the created image representations with polarization contrast by means of the imaging apparatus in a manner overlaid with white-light image representations captured by means of at least two video cameras for capturing multicolored visible light.

3. The visualization arrangement as claimed in claim 1,
wherein
at least one polarization-filtering device is arranged in the beam path downstream of the illumination device and upstream of an object space region.

4. The visualization arrangement as claimed in claim 1,
wherein
at least one of the polarization-filtering devices comprises a plurality of polarizers that differ from one another.

5. The visualization arrangement as claimed in claim 1,
wherein
the at least one further video camera is designed to capture monochrome light, or at least one of the aforementioned video cameras takes the form of a polarization camera.

6. The visualization arrangement as claimed in claim 5,
wherein
at least one polarization-filtering device arranged upstream of a video camera for capturing multicolored visible light comprises a circular polarizer.

7. The visualization arrangement as claimed in claim 1,
wherein
the evaluation device is designed to use the images captured by the video cameras to determine a set of Mueller matrix coefficients and to determine a polarization contrast derived from said coefficients.

8. The visualization arrangement as claimed in claim 1,
wherein
a polarization-filtering device arranged in the beam path downstream of the illumination device and upstream of an object space region is configured to simultaneously polarize at least two defined, mutually deviating wavelengths in a manner deviating from one another.

9. The visualization arrangement as claimed in claim 1,
wherein
at least one of the polarization-filtering devices comprises a wavelength filter and/or a wavelength-selective polarizer.

10. The visualization arrangement as claimed in claim 1,
wherein
the illumination device is designed to emit bichromatic light.

11. The visualization arrangement as claimed in claim 1,
wherein
the illumination device is designed to emit polychromatic light, and a polarization-filtering device arranged in the beam path downstream of the illumination device and upstream of an object space region comprises at least one notch filter and/or a number of bandpass filters.

12. The visualization arrangement as claimed in claim 1,
characterized by at least one beam splitter that is arranged in the beam path between an object space region and the video cameras.

13. A visualization arrangement for microsurgery, comprising an imaging apparatus, an illumination device and a polarization-determining arrangement,
wherein
the polarization-determining arrangement comprises two polarization cameras, with each of the two polarization cameras being assigned an individual partial beam path in the beam path, and an evaluation device,
wherein the evaluation device is designed to create image representations with polarization contrast using the images captured by the polarization cameras and to display said image representations by means of the imaging apparatus,
wherein the evaluation device is designed to display the created image representations with polarization contrast by means of the imaging apparatus in a manner overlaid with white-light image representations captured by means of the two polarization cameras.

14. (canceled)

15. A method for creating an image representation with polarization contrast of an object by a visualization arrangement for microsurgery as claimed in claim 1,
wherein
the method comprising the following steps:
radiating light waves onto the object by means of the illumination device,
capturing light waves, which are emitted by the object following an interaction of the object with the radiated-in light waves, by means of the at least three video cameras or the at least two polarization cameras,
creating an image representation with polarization contrast on the basis of a generated polarization state of the radiated-in light waves and/or an analyzed polarization state of the captured light waves.

16. The method as claimed in claim 15,
wherein
light waves in a defined polarization state are radiated-in by means of the illumination device and a polarization-filtering device arranged in the beam path downstream of the illumination device and upstream of the object.

17. The method as claimed in claim 15,
wherein
the polarization state of the received light waves is analyzed by means of the at least three video cameras and the polarization-filtering devices arranged upstream of said video cameras in the beam path and/or by means of the polarization cameras.

18. The method as claimed in claim 15,
wherein
the evaluation device is used to create an image representation with polarization contrast on the basis of an analyzed polarization state of the captured light waves, wherein a set of Mueller matrix coefficients is determined by means of the images captured by the video cameras and/or the polarization cameras, and a polarization contrast derived from said coefficients is determined.

19. The method as claimed in claim 15,
wherein
the image representation with polarization contrast is created within a time interval of less than 50 ms, and/or the resolution of the stereoscopic image representation with polarization contrast created deviates from the resolution of a corresponding image representation without polarization contrast by less than 10 percent.

20. The method as claimed in claim 19,
wherein
the image representation with polarization contrast is created within a time interval of less than 50 ms and displayed in a manner overlaid on a multicolored image representation.

21. A microscope comprising a visualization arrangement as claimed in claim 1.
